(19) United States
(12) Patent Application Publication
(10) Pub. No.: US 2026/0345865 A1
(43) Pub. Date: Nov. 5, 2026

(54) DEVELOPMENT OF A DURAL SUBSTITUTE BIOMATERIAL USING HUMAN DURADERIVED STEM CELLS AND A DURAL SUBSTITUTE PREPARED THEREFROM

(71) Applicants: THE CATHOLIC UNIVERSITY OF KOREA INDUSTRY-ACADEMIC COOPERATION FOUNDATION, Seoul (KR); GACHON UNIVERSITY OF INDUSTRY-ACADEMIC COOPERATION FOUNDATION, Gyeonggi-do (KR)

(72) Inventors: Sun Hwa PARK, Seoul (KR); Sung Won KIM, Seoul (KR); Seung Ho YANG, Seoul (KR); Hyun Ji LEE, Seoul (KR); Jin Woo LEE, Gyeonggi-do (KR); Young Soo YU, Gyeonggi-do (KR)

(21) Appl. No.: 19/663,836

(22) Filed: Apr. 30, 2026

(30) Foreign Application Priority Data
Apr. 30, 2025 (KR) 10-2025-0057617

(51) Int. Cl. A61L 27/38; A61L 27/18

(52) U.S. Cl. CPC A61L 27/3878; A61L 27/18; A61L 27/3834; A61L 2430/32

(57) ABSTRACT

The present invention relates to the development of a dural substitute biomaterial using human dura-derived stem cells and to a dural substitute prepared therefrom. Specifically, dural substitutes in the form of nanofiber sheets of various sizes and shapes were manufactured through an electrospinning process using polymers. When the electrospun surface of the dural substitutes of the present invention were treated with dura mater-derived stem cells, the stem cells exhibited significantly good adhesion and engraftment abilities as well as high angiogenesis ability. Therefore, a method and conditions for manufacturing dural substitutes have been established, and the dural substitute manufacturing method and the dural substitutes manufactured therefrom are expected to be effectively used.

### CROSS-REFERENCE TO RELATED APPLICATION

This application claims priority to and the benefit of Korean Patent Application No. 10-2025-0057617, filed on Apr. 30, 2025, the disclosure of which is incorporated herein by reference in its entirety.

### TECHNICAL FIELD

The present invention relates to the development of a dural substitute biomaterial using human dura-derived stem cells and a dural substitute prepared therefrom.

### BACKGROUND ART

The dura mater, which is the outermost layer of the meninges that surround and protect the brain and spinal cord, is composed of strong and tough connective tissue. The dura mater provides structural support within the cranial and spinal canals and protects the brain and spinal cord along with the inner pia mater and arachnoid mater. Furthermore, the dura mater is closely connected to the skull and spine, serving as a crucial barrier that protects the central nervous system from external impacts.

One of the primary functions of the dura mater is to protect the central nervous system from mechanical damage. Since the dura mater is composed of thick connective tissue, it withstands external pressure or impact and serves as a barrier to prevent damage to internal tissues. Furthermore, by forming a space containing cerebrospinal fluid, the meninges provide buoyancy and serve as a buffer, and thus help prevent the brain from rubbing directly against the skull.

In addition, the dura mater maintains the homeostasis of the central nervous system through its vascular and nerve distribution. The venous sinuses within the dura mater serve to return blood drained from the brain to the heart, and the presence of immune cells contributes to defense against infection. Recent studies have revealed that the dura mater plays a crucial role in maintaining nervous system health by participating in the exchange of substances between the cerebrospinal fluid and the lymphatic system.

When dural damage or defects occurs, the risk of cerebrospinal fluid leakage, infection, and nerve damage increases, and thus dural substitutes materials for restoration are necessary. Conventional autologous tissue transplantation has a limited supply and the burden of additional surgery, while allogeneic or xenogeneic transplantation carries risks of immune reactions and infection.

Despite the importance of the dura mater, there are hardly any reported dural substitute materials and dural substitutes manufactured therefrom.

### PRIOR ART DOCUMENTS

### Patent Document

(Korean Patent Publication) No. 10-2020-0051077.

### DISCLOSURE

### Technical Problem

One object of the present invention is to provide a method of manufacturing a dural substitute, comprising the following steps, wherein the dural substitute is a nanofiber assembly:
S1) a step of preparing a composition for manufacturing a dural substitute by dissolving a polymer in a solvent and manufacturing a dural substitute by electrospinning the composition; and
S2) a step of treating the electrospun surface of the dural substitute with dura-derived stem cells.

Another object of the present invention is to provide a dural substitute manufactured by the above-described method.

Yet another object of the present invention is to provide a composition for manufacturing a dural substitute, comprising a polymer and a solvent as active ingredients,
wherein the dural substitute is a nanofiber assembly.

Yet another object of the present invention is to provide a kit for manufacturing a dural substitute, comprising the above-described composition for manufacturing a dural substitute and instructions.

However, technical problems to be solved in the present invention are not limited to the above-described problems, and other problems which are not described herein will be fully understood by those of ordinary skill in the art from the following descriptions.

### Technical Solution

The present invention provides a method of manufacturing a dural substitute, comprising:
S1) a step of preparing a composition for manufacturing a dural substitute by dissolving a polymer in a solvent and manufacturing a dural substitute by electrospinning the composition; and
S2) a step of treating the electrospun surface of the dural substitute with dura-derived stem cells,
wherein the dural substitute is a nanofiber assembly.

In one embodiment of the present invention, wherein the thickness of the nanofiber and the pore size within the nanofiber assembly may be characterized as being 0.1 μm to 10 μm, but is not limited thereto.

In one embodiment of the present invention, wherein the nanofiber assembly may be any one selected from the group consisting of a nanofiber sheet, a nanofiber mat, a nanofiber film, a nanofiber membrane, a nanofiber web, a nanofiber textile, and a nanofiber fabric, but is not limited thereto.

In one embodiment of the present invention, wherein the polymer may be any one selected from the group consisting of polycaprolactone (PCL), polyurethane (PU), a poly(lactic-co-glycolic acid) (PLGA) copolymer, polylactic acid (PLA), poly(3-hydroxybutyrate-co-3-hydroxyvalerate) (PHBV), polyvinyl alcohol (PVA), collagen, gelatin, elastin, chitosan, silk fibroin, alginate, poly(glycolic acid) (PGA), poly(L-lactic acid) (PLLA), poly(L-lactic acid)/collagen (PLLA/CL), and polyethylene oxide (PEO), but is not limited thereto.

In one embodiment of the present invention, wherein the solvent may be any one selected from the group consisting of formic acid, chloroform acid, acetic acid, acetone, dichloromethane, 1,2-dichloroethane, dimethylformamide, ethanol, isopropyl alcohol, water, and tetrahydrofuran, but is not limited thereto.

In one embodiment of the present invention, wherein the electrospinning may be characterized by the following conditions, but is not limited thereto:
a voltage of 1 to 30 kV;
a flow rate of 0.1 to 5 mL/h; and
a distance of 1 to 20 cm between a nozzle and a collector.

In one embodiment of the present invention, the method may further comprise, S3) a step of treating the electrospun surface of the dural substitute with an angiogenic cell, but is not limited thereto.

In one embodiment of the present invention, wherein the angiogenic cell may be any one selected from the group consisting of a stem cell, an induced pluripotent stem cell, a vascular endothelial cell, and an human umbilical vein endothelial cell (HUVEC), but is not limited thereto.

The present invention provides a dural substitute manufactured by the above-described method.

The present invention provides a composition for manufacturing a dural substitute, comprising a polymer and a solvent as active ingredients, wherein the dural substitute is a nanofiber assembly.

In one embodiment of the present invention, wherein the polymer may be any one selected from the group consisting of polycaprolactone (PCL), polyurethane (PU), a poly(lactic-co-glycolic acid) (PLGA) copolymer, polylactic acid (PLA), poly(3-hydroxybutyrate-co-3-hydroxyvalerate) (PHBV), polyvinyl alcohol (PVA), collagen, gelatin, elastin, chitosan, silk fibroin, alginate, poly(glycolic acid) (PGA), poly(L-lactic acid) (PLLA), poly(L-lactic acid)/collagen (PLLA/CL), and polyethylene oxide (PEO), but is not limited thereto.

In one embodiment of the present invention, wherein the solvent may be any one selected from the group consisting of formic acid, chloroform acid, acetic acid, acetone, dichloromethane, 1,2-dichloroethane, dimethylformamide, ethanol, isopropyl alcohol, water, and tetrahydrofuran, but is not limited thereto.

The present invention provides a kit for manufacturing a dural substitute, comprising the composition for manufacturing a dural substitute and instructions.

In one embodiment of the present invention, wherein the instructions may teach the method of any one of the above claims, but is not limited thereto.

Additionally, the present invention provides a use of the composition in the manufacture of a dural substitute.

Additionally, the present invention provides a use of the composition for manufacturing a dural substitute.

### Advantageous Effects

According to the development of a dural substitute biomaterial using human dura-derived stem cells and a dural substitute prepared therefrom, a dural substitute composed of nanofibers having various thicknesses and shapes was manufactured through an electrospinning process using a polymer. When the electrospun surface of the dural substitute of the present invention was treated with dura-derived stem cells, the stem cells exhibited not only significantly excellent adhesion ability and engraftment ability, but also high angiogenesis ability in the stem cells, thereby establishing a method and conditions for manufacturing a dural substitute, and thus the method of manufacturing a dural substitute and the dural substitute manufactured therefrom are expected to be usefully utilized.

### DESCRIPTION OF DRAWINGS

FIG. 1 shows a process of manufacturing a dural substitute through an electrospinning process of the present invention.

FIG. 2A shows dural substitutes manufactured with nanofibers of various sizes through an electrospinning process.

FIG. 2B shows dural substitutes manufactured with nanofibers having thicknesses of 0.7 μm and 4 μm through an electrospinning process using PCL.

FIG. 2C shows, respectively, dural substitutes manufactured with nanofibers having a thickness of 0.7 μm through an electrospinning process using PCL and PU.

FIG. 3A shows human dura-derived stem cells.

FIG. 3B shows results confirming adhesion ability and engraftment ability of the dura-derived stem cells when the dura-derived stem cells were treated on the dural substitute.

FIGS. 4A to 4C show results confirming angiogenesis ability in the dura-derived stem cells attached to the electrospun surface of dural substitute.

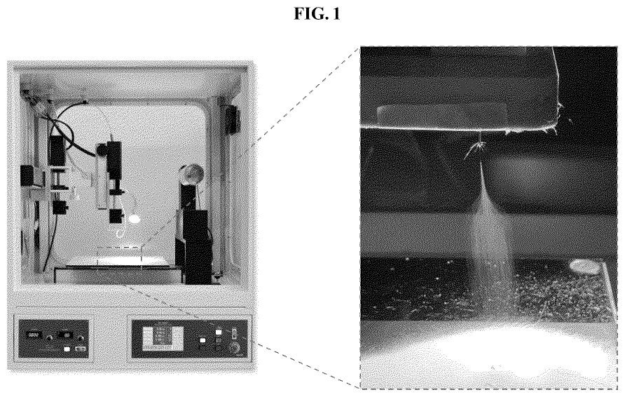

### MODES OF THE INVENTION

The present invention provides a method of manufacturing a dural substitute, comprising:
S1) a step of preparing a composition for manufacturing a dural substitute by dissolving a polymer in a solvent and manufacturing a dural substitute by electrospinning the composition; and
S2) a step of treating the electrospun surface of the dural substitute with dura-derived stem cells,
wherein the dural substitute is a nanofiber assembly.

In the present invention, the “dura mater” is the outermost tough membrane that surrounds and protects the brain and spinal cord. The meninges are composed of the dura mater, the arachnoid mater, and the pia mater, among which the dura mater is the thickest and strongest. The dura mater protects the brain and spinal cord from external impacts, and also serves to regulate the flow of cerebrospinal fluid. In particular, the dura mater forms dural venous sinuses within the cranial cavity and plays an important role in venous blood drainage.

Since the dura mater plays a strong structural role in protecting the brain and spinal cord, it is not a simple membrane but a special tissue that should satisfy both biocompatibility and strength. A substitutes material should have low immunogenicity and sufficient tensile strength, but at the same time, there is a difficulty in that the substitutes material should also be flexible. Additionally, the leakage prevention function and the cerebrospinal fluid blocking function should be maintained, and the substitutes may cause inflammation or adhesion. For these reasons, various attempts have been made using autologous tissue (autologous membrane), xenogeneic tissue (animal membrane), artificial materials, and the like, but it is difficult to develop a perfect substitutes. However, in one embodiment of the present invention, a nanofiber assembly was manufactured by electrospinning the composition of the present invention, and it was confirmed that the nanofiber assembly exhibits excellent functions as a dura mater.

In the present invention, the “nanofiber assembly” refers to a three-dimensional structure formed by a plurality of nanofibers having thicknesses of several tens of nanometers to several hundreds of nanometers. The nanofibers may be entangled with, crossed with, or laminated on one another to form a continuous or discontinuous network structure. Additionally, the nanofiber assembly may exhibit various physical properties, including porosity, mechanical strength, and surface area characteristics. In the present specification, the nanofiber assembly is defined as a concept encompassing all forms of assembled nanofibers having such structural and physical characteristics.

In one embodiment of the present invention, the dural substitute manufactured as a nanofiber assembly was in the form of a nanofiber sheet, but in one embodiment of the present invention, wherein the nanofiber assembly may be any one selected from the group consisting of a nanofiber sheet, a nanofiber mat, a nanofiber film, a nanofiber membrane, a nanofiber web, a nanofiber textile, and a nanofiber fabric, but is not limited thereto, and it is obvious that any one of the above-described forms may exhibit the characteristics as a dural substitute confirmed in the nanofiber sheet of the present invention.

In the present invention, the “nanofiber mat” may refer to a thin membrane formed by entangled nonwoven nanofibers. The nanofiber mat may have characteristics compatible with a “nanofiber sheet,” and the expression “mat” may be used when emphasizing a thicker and softer texture, but is not necessarily limited thereto.

In the present invention, the “nanofiber film” may refer to a membrane that is thinner and more uniform than a sheet. The nanofiber film may be manufactured in combination with a solution-based coating process, such as solvent casting or hot pressing, but is not limited thereto.

In the present invention, the “nanofiber membrane” may refer to a thin structure, such as a filter having a selective permeation function, and may be mainly used for a separator or filtration application. The nanofiber membrane may be characterized by a finer pore structure and specific functionality, such as filtration or ion exchange, than a general “sheet,” but is not necessarily limited thereto.

In the present invention, the “nanofiber web” may refer to a structure formed by entangled electrospun nanofibers, and may mean a looser and softer form than a “sheet.”

In the present invention, the “nanofiber textile or fabric” may be a general term referring to a fiber product manufactured by processing nanofibers into a yarn form and then producing the same in a woven or nonwoven form.

At least one selected from the group consisting of the nanofiber sheet, the nanofiber mat, the nanofiber film, the nanofiber membrane, the nanofiber web, the nanofiber textile, and the nanofiber fabric described above as examples of the nanofiber assembly may be one form of various types of nanofiber assemblies having characteristics at the same level as those of the nanofiber sheet.

In one embodiment of the present invention, wherein the thickness of the nanofiber and the pore size of the nanofiber assembly may be characterized as being 0.1 μm to 10 μm, but is not limited thereto. For example, the thickness of the nanofiber may be 0.1 μm to 9 μm, 0.1 μm to 8 μm, 0.1 μm to 7 μm, 0.1 μm to 6 μm, 0.1 μm to 5 μm, 0.1 μm to 4 μm, 0.3 μm to 10 μm, 0.3 μm to 9 μm, 0.3 μm to 8 μm, 0.3 μm to 7 μm, 0.3 μm to 6 μm, 0.3 μm to 5 μm, 0.3 μm to 4 μm, 0.5 μm to 10 μm, 0.5 μm to 9 μm, 0.5 μm to 8 μm, 0.5 μm to 7 μm, 0.5 μm to 6 μm, 0.5 μm to 5 μm, 0.5 μm to 4 μm, 0.7 μm to 10 μm, 0.7 μm to 9 μm, 0.7 μm to 8 μm, 0.7 μm to 7 μm, 0.7 μm to 6 μm, 0.7 μm to 5 μm, 0.7 μm to 4 μm, or 4 μm, but is not limited thereto. Alternatively, the thickness of the nanofiber may be 0.1 μm to 3.5 μm, 0.1 μm to 3 μm, 0.1 μm to 2.5 μm, 0.1 μm to 2 μm, 0.1 μm to 1.5 μm, 0.1 μm to 1 μm, 0.1 μm to 0.9 μm, 0.1 μm to 0.8 μm, 0.1 μm to 0.7 μm, 0.3 μm to 3.5 μm, 0.3 μm to 3 μm, 0.3 μm to 2.5μ, 0.3 μm to 2 μm, 0.3 μm to 1.5 μm, 0.3 μm to 1 μm, 0.3 μm to 0.9 μm, 0.3 μm to 0.8 μm, 0.3 μm to 0.7 μm, 0.5 μm to 3.5 μm, 0.5 μm to 3 μm, 0.5 μm to 2.5 μm, 0.5 μm to 2 μm, 0.5 μm to 1.5 μm, 0.5 μm to 1 μm, 0.5 μm to 0.9 μm, 0.5 μm to 0.8 μm, 0.5 μm to 0.7 μm, or 0.7 μm, but is not limited thereto.

In the present invention, the “thickness of the nanofiber” means a diameter measured at a cross-section of the nanofiber, and may be used interchangeably with the “diameter of the nanofiber,” but is not limited thereto. The thickness of the nanofiber directly affects the surface area, mechanical strength, and cell adhesion characteristics of the fiber. In particular, as the thickness decreases, the surface area per unit area increases, thereby providing an environment favorable for cell adhesion and proliferation. However, an excessively small thickness may reduce the mechanical stability of the fiber, thereby causing structural collapse when used as a tissue engineering substitutes. Conversely, when the thickness is excessively large, the unit surface area decreases, and thus there is a concern that interaction with cells may be hindered. Therefore, in manufacturing a tissue engineering substitutes, setting the thickness of the nanofiber within an appropriate may be essential for simultaneously securing physical properties and biocompatibility.

In one embodiment of the present invention, when the thickness of the nanofiber was 0.7 μm, the spacing between the nanofibers was narrow, the adhesion area was large, and the nanofibers were evenly distributed, such that interactions with stem cells and angiogenic cells were actively achieved, and accordingly, cell adhesion ability, viability, and angiogenesis level on the nanofiber sheet were all excellent, thereby confirming that the nanofiber sheet exhibits remarkably excellent suitability as a dural substitute. In addition, even when the thickness of the nanofiber was 4 μm, it was confirmed that dura-derived stem cells were attached at a high level and vascular structures were formed, similarly to the nanofiber assembly having a thickness of 0.7 μm. Accordingly, it was demonstrated that the dural substitute manufactured using the nanofiber assembly having a thickness of 0.7 μm to 4 μm exhibits excellent characteristics in terms of cell affinity, mechanical stability, and pore structure.

Additionally, the pore size of the nanofiber assembly may be 0.1 μm to 10 μm, 0.1 μm to 9 μm, 0.1 μm to 8 μm, 0.1 μm to 7 μm, 0.1 μm to 6 μm, 0.1 μm to 5 μm, 0.1 μm to 4 μm, 0.3 μm to 10 μm, 0.3 μm to 9 μm, 0.3 μm to 8 μm, 0.3 μm to 7 μm, 0.3 μm to 6 μm, 0.3 μm to 5 μm, 0.3 μm to 4 μm, 0.5 μm to 10 μm, 0.5 μm to 9 μm, 0.5 μm to 8 μm, 0.5 μm to 7 μm, 0.5 μm to 6 μm, 0.5 μm to 5 μm, 0.5 μm to 4 μm, 0.7 μm to 10 μm, 0.7 μm to 9 μm, 0.7 μm to 8 μm, 0.7 μm to 7 μm, 0.7 μm to 6 μm, 0.7 μm to 5 μm, or 0.7 μm to 4 μm, but is not limited thereto.

In the present invention, the “pore within the nanofiber” means a fine empty space or gap formed inside the nanofiber itself or between the nanofibers, and the pore may generally have a size of several tens of nanometers (nm) to several hundreds of nanometers (nm). The thickness of the nanofiber, that is, the fiber diameter, is a factor that significantly affects the formation of a porous architecture within the sheet, and the pore size in an electrospinning-based nanofiber sheet is determined by the fiber diameter and the spacing between the fibers. In general, when the fiber diameter is too small, the spacing between the fibers becomes narrow, such that the pore size becomes small, which may be unfavorable for cell infiltration and body fluid exchange, whereas when the diameter is too large, the surface area decreases, which may cause a problem in that cell adhesion efficiency decreases.

In one embodiment of the present invention, as a result of analyzing the size of the pores within the sheet of the nanofiber sheet having fiber diameters of 0.7 μm and 4 μm through an electrospinning process, it was confirmed that the pore size of the nanofiber assembly was 0.7 μm and 4 μm, and it was confirmed that the most excellent physical and biological functionality was exhibited within this range.

Therefore, in one embodiment of the present invention, the thickness of the nanofiber sheet and the size of the pores within the nanofiber may be 0.7 μm to 4 μm, but is not limited thereto.

In one embodiment of the present invention, wherein the polymer may be any one selected from the group consisting of polycaprolactone (PCL), polyurethane (PU), a poly(lactic-co-glycolic acid) (PLGA) copolymer, polylactic acid (PLA), poly(3-hydroxybutyrate-co-3-hydroxyvalerate) (PHBV), polyvinyl alcohol (PVA), collagen, gelatin, elastin, chitosan, silk fibroin, alginate, poly(glycolic acid) (PGA), poly(L-lactic acid) (PLLA), poly(L-lactic acid)/collagen (PLLA/CL), and polyethylene oxide (PEO), but is not limited thereto, and may further additionally comprise a composite polymer mixed with a natural polymer such as gelatin, collagen, or chitosan.

In one embodiment of the present invention, polycaprolactone (hereinafter, PCL) was used as one example of the polymer, and characteristics thereof are as follows, but are not limited thereto:
(1) Biocompatibility: suitable as a biomaterial because it does not react significantly with the human body;
(2) Biodegradability: degraded over several months to several years, and suitable for tissue regeneration purposes;
(3) Mechanical properties: provides flexibility and elasticity due to a low glass transition temperature (Tg); and
(4) Processability: suitable for 3D printing due to a low melting point (~60 °C).

In one embodiment of the present invention, polyurethane (hereinafter, PU) was used as one example of the polymer, and characteristics thereof are as follows, but are not limited thereto:
(1) Mechanical strength: excellent structural stability due to high tensile strength and durability;
(2) Elasticity and flexibility: applicable in various environments due to excellent stretchability and resilience;
(3) Chemical resistance: high resistance to water, organic solvents, heat, and abrasion; and
(4) Biocompatibility: may be utilized for medical use because biocompatibility may be adjustable depending on the composition.

According to one embodiment of the present invention, it was confirmed that, when PCL and PU are used, a dural substitute in the form of a nanofiber assembly characterized by the thickness of the nanofiber of the present invention and the pore size within the thickness of the nanofiber may be successfully manufactured. At this time, the dural substitute using both polymers may exhibit characteristics centered on cell affinity (PCL-based) or centered on mechanical support (PU-based), respectively. However, any one of these respective characteristics is not necessarily suitable or required as a dural substitute, and when structural characteristics including the thickness of the nanofiber of the present invention and the pore size within the thickness of the nanofiber are provided, application as a dural substitute may be possible due to cell-friendly surface characteristics and biodegradability, regardless of what the main characteristics are.

In one embodiment of the present invention, formic acid was used as a solvent when the thickness of the nanofiber was 0.5 to 2 μm, and chloroform was used as a solvent when the thickness of the nanofiber was 4 to 20 μm. When the solvents are different as described above, such selection may be made for the purpose of fiber formation stability and viscosity control to induce the thickness of the nanofiber to a target value, but is not necessarily limited thereto. That is, when electrospinning may be uniformly performed to the thickness of the nanofiber confirmed to be excellent in the present invention, a suitable solvent may be used according to the type of polymer. For example, the viscosity of formic acid is 1.804 cP and the viscosity of chloroform is 0.563 cP, such that there is a viscosity difference of about three times or more, and such a viscosity difference may directly affect the fluidity and evaporation rate of the solution during electrospinning. Formic acid has high viscosity and a low evaporation rate, such that the jet is stretched further during spinning, and may be suitable for forming thin, long, and uniform fibers (0.5 to 2 μm), but is not limited thereto. Additionally, chloroform may rapidly solidify during spinning due to low viscosity and high volatility, and because the time for stretching is relatively short, may be suitable for forming thick and coarse fibers (4 to 20 μm), but is not limited thereto. Therefore, when the characteristics of each polymer are considered by applying such criteria, it may be readily understood that a dural substitute characterized by the thickness of the nanofiber of the present invention may be manufactured even by using a solvent that may be generally used in the art.

Meanwhile, in one embodiment of the present invention, an electrospinning process was performed using PCL having an average molecular weight of 80,000 g/mol supplied by Sigma-Aldrich and Tecoflex® EG-80A of Lubrizol (TPU, average molecular weight of about 145,000 g/mol, aliphatic polyether type). The average molecular weights of the two polymers may be values selected in consideration of processability and process stability for manufacturing each polymer into fiber diameters having thicknesses of 0.7 μm and 4 μm through the electrospinning process, but are not limited thereto.

Therefore, in one embodiment of the present invention, the solvent may be any one selected from the group consisting of formic acid, chloroform acid, acetic acid, acetone, dichloromethane, 1,2-dichloroethane, dimethylformamide, ethanol, isopropyl alcohol, water, and tetrahydrofuran, but is not limited thereto.

In one embodiment of the present invention, wherein the electrospinning may be characterized by the following conditions, but is not limited thereto:
a voltage of 1 to 30 kV;
a flow rate of 0.1 to 5 mL/h; and
a distance of 1 to 20 cm between a nozzle and a collector.

In the present invention, the voltage may be 1 to 30 kV, 1 to 29.5 kV, 1 to 29 kV, 1 to 28.5 kV, 1 to 28 kV, 1 to 27.5 kV, 1 to 27 kV, 1 to 26.5 kV, 3 to 30 kV, 3 to 29.5 kV, 3 to 29 kV, 3 to 28.5 kV, 3 to 28 kV, 3 to 27.5 kV, 3 to 27 kV, 3 to 26.5 kV, 5 to 30 kV, 5 to 29.5 kV, 5 to 29 kV, 5 to 28.5 kV, 5 to 28 kV, 5 to 27.5 kV, 5 to 27 kV, 5 to 26.5 kV, 7 to 30 kV, 7 to 29.5 kV, 7 to 29 kV, 7 to 28.5 kV, 7 to 28 kV, 7 to 27.5 kV, 7 to 27 kV, or 7 to 26.5 kV, but is not limited thereto.

In the present invention, the flow rate may be 0.1 to 5 mL/h, 0.1 to 4 mL/h, 0.1 to 3 mL/h, 0.1 to 2 mL/h, 0.1 to 1 mL/h, 0.1 to 0.5 mL/h, 0.3 to 5 mL/h, 0.3 to 4 mL/h, 0.3 to 3 mL/h, 0.3 to 2 mL/h, 0.3 to 1 mL/h, 0.3 to 0.5 mL/h, 0.5 to 5 mL/h, 0.5 to 4 mL/h, 0.5 to 3 mL/h, 0.5 to 2 mL/h, 0.5 to 1 mL/h, or 0.5 mL/h, or 1 mL/h, but is not limited thereto.

In the present invention, the distance between the nozzle and the collector may be 1 to 20 cm, 1 to 15 cm, 1 to 13 cm, 1 to 10 cm, 5 to 20 cm, 5 to 15 cm, 5 to 13 cm, 5 to 10 cm, 7 to 20 cm, 7 to 15 cm, 7 to 13 cm, 7 to 10 cm, 10 to 20 cm, 10 to 15 cm, 10 to 13 cm, or 10 cm, but is not limited thereto.

In electrospinning, the voltage may directly affect the tensile force of the spinning stream and the fiber diameter, and in one embodiment of the present invention, voltage range conditions optimized for a target value of the thickness of each nanofiber were explored, and 7 kV to 26.5 kV was set as a range in which fiber uniformity and shape stability were simultaneously secured.

Meanwhile, in electrospinning, in general, as the flow rate increases, the fibers become thicker, and when the flow rate is too low, fiber breakage may occur. As with the voltage, in one embodiment of the present invention, conditions of a voltage range optimized for a target value of the thickness of each nanofiber were explored and derived, and for example, under a condition for forming 4 μm fibers, the most uniform and stable formation result may be exhibited when the flow rate is 0.5 mL/h.

That is, the electrospinning conditions (formic acid/chloroform, voltage, and flow rate) of the present invention may all be experimentally designed conditions for precisely controlling the fiber diameter, and may be substantial constituent elements for a technical effect. As described above, in the present invention, since the polymer and solvent conditions may vary depending on the desired thickness of the nanofiber, it may be understood that the voltage condition, the flow rate condition, and the distance condition between the nozzle and the collector used in one embodiment of the present invention may also vary, and that the function as a dural substitute does not vary depending on the voltage condition.

In one embodiment of the present invention, the manufacturing method may further comprise, S3) a step of treating the electrospun surface of the dural substitute with an angiogenic cell, but is not limited thereto.

In one embodiment of the present invention, wherein the angiogenic cell may be any one selected from the group consisting of a stem cell, an induced pluripotent stem cell, a vascular endothelial cell, and an human umbilical vein endothelial cell (HUVEC), but is not limited thereto.

The present invention provides a dural substitute manufactured by the above-described method.

In the present invention, as an artificial or biologically derived material used to supplement or regenerate a damaged or defective dura mater, the dural substitute of the present invention may mean a nanofiber assembly manufactured by electrospinning the composition for manufacturing a dural substitute of the present invention.

The present invention provides a composition for manufacturing a dural substitute, comprising a polymer and a solvent as active ingredients, wherein the dural substitute is a nanofiber assembly.

In one embodiment of the present invention, wherein the polymer may be any one selected from the group consisting of polycaprolactone (PCL), polyurethane (PU), polyvinyl alcohol (PVA), collagen, gelatin, elastin, chitosan, silk fibroin, alginate, poly(glycolic acid) (PGA), poly(lactic-co-glycolic acid) (PLGA), poly(L-lactic acid) (PLLA), poly(L-lactic acid)/collagen (PLLA/CL), and polyethylene oxide (PEO), but is not limited thereto.

In one embodiment of the present invention, wherein the solvent may be any one selected from the group consisting of formic acid, chloroform acid, acetic acid, acetone, dichloromethane, 1,2-dichloroethane, dimethylformamide, ethanol, isopropyl alcohol, water, and tetrahydrofuran, but is not limited thereto.

The present invention provides a kit for manufacturing a dural substitute, comprising the composition for manufacturing a dural substitute and instructions.

In one embodiment of the present invention, wherein the instructions may teach the method of any one of the above claims, but are not limited thereto.

In the present invention, the “kit” means a tool that enables manufacture of a dural substitute using the composition for manufacturing a dural substitute of the present invention. In the kit of the present invention, in addition to the above materials, other components, compositions, solutions, devices, and the like that are commonly required for storage and treatment thereof may be included. As a specific example, each constituent may be applied one or more times without limitation on the number of times, there is no limitation on the order of applying each material, and application of each material may be performed simultaneously or sequentially.

In the present invention, the kit may comprise a container, instructions, and the like. The container may serve to package the material, and may also serve to store and secure the material. The container may, for example, take the form of a bottle, a tub, a sachet, an envelope, a tube, an ampoule, or the like, and these may be formed partially or entirely from plastic, glass, paper, foil, wax, or the like. The container may be fitted with a fully or partially removable cap that is initially a part of the container or may be attached to the container by mechanical, adhesive, or other means, and may also be fitted with a stopper through which the contents may be accessed by a needle. The kit may comprise an outer package, and the outer package may comprise instructions regarding use of the components.

Hereinafter, preferred examples are presented to help understand the present invention. However, the following examples are provided only to help understand the present invention more easily, and the contents of the present invention are not limited by the following examples.

### EXAMPLES

### Example 1. Manufacture of Dural Substitutes Via Electrospinning

Dural substitutes in the form of nanofiber assemblies were manufactured using an electrospinning method. Polycaprolactone (PCL) and polyurethane (PU), which are polymers, were used as main materials. Each polymer was prepared as a solution according to Table 1, and an electrospinning process was performed under each set of conditions. At this time, PU was used under the same conditions as PCL in Table 1.

TABLE 1

| Fiber | Concentration | | | Flow | |
|---|---|---|---|---|---|
| diameter | of PCL | | Voltage | rate | Distance |
| (μm) | (w/v %) | Solvent | (kV) | (mL/h) | (cm) |
| 0.5 | 15 | Formic acid | 25 | 1.0 | 10 |
| 0.7 | 20 | | 25 | | |
| 1 | 30 | | | | |
| 1.5 | 35 | | 23 | | |
| 2 | 40 | | 10 | | |
| 4 | 15 | Chloroform | 26.5 | 0.5 | |
| 7 | 25 | | 8.5 | 1.0 | |
| 10 | 25 | | 7 | | |
| 20 | 40 | | 7.5 | | |

### Example 2. Characterization of Dural Substitute

In Example 1, the dural substitutes were manufactured through an electrospinning process. In Example 2, various characteristics of the dural substitutes were analyzed based on the thickness of the nanofibers constituting the dural substitutes manufactured in Example 1 and the type of polymer.

By adopting and verifying some of the electrospinning conditions listed in Table 1 of Example 1, dural substitutes in the form of a uniform nanofiber sheet were manufactured as an assembly of nanofibers.

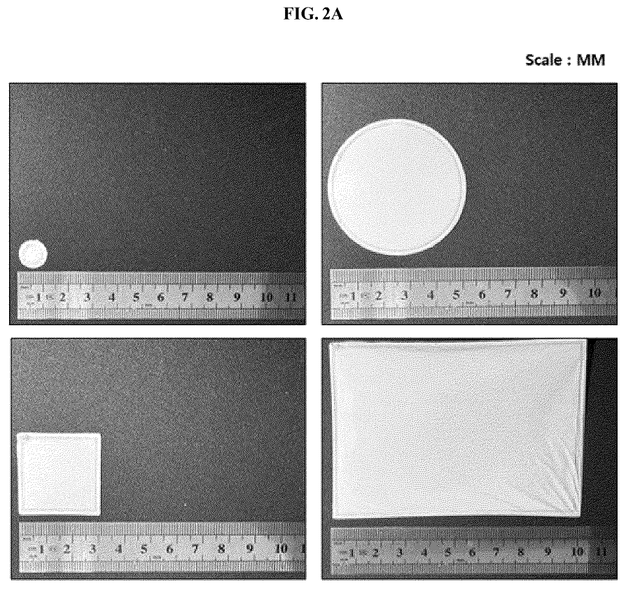

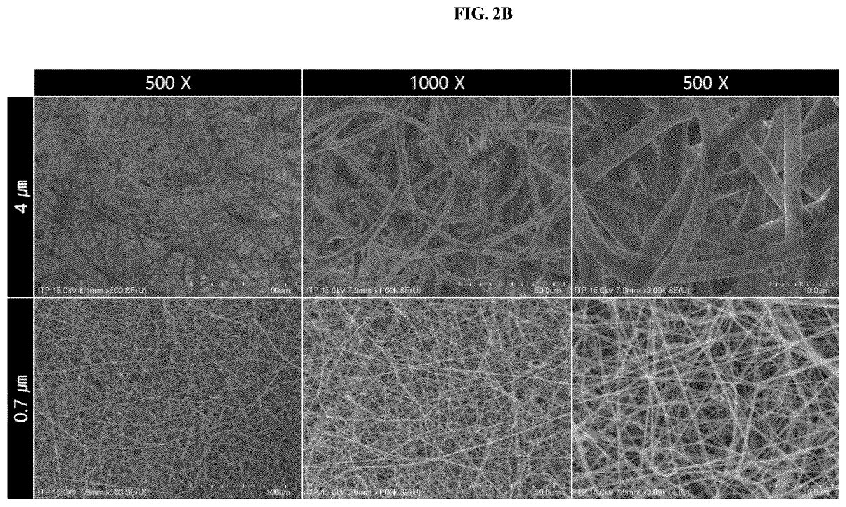

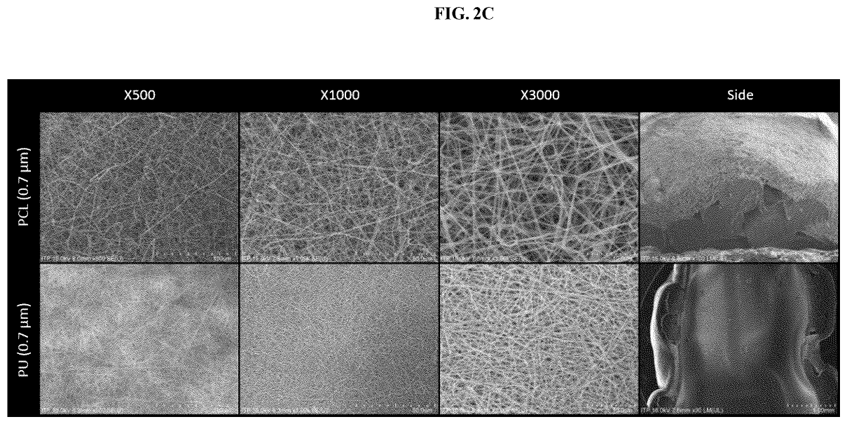

The results confirmed that the polymers used to manufacture the dural substitutes and the electrospinning process conditions utilizing the polymers enable not only control of the fiber diameter and structure in various ways but also the manufacture of sheets of various shapes based on representative conditions (FIG. 2A).

Furthermore, the dural substitutes having a nanofiber thickness of 0.7 μm and 4 μm and formed using the polymer PCL were adopted to verify the fiber structural thickness and the resulting functional effects and were observed under magnification using a scanning electron microscope (SEM). Specifically, two types of nanofiber sheets manufactured under conditions of nanofiber thicknesses of 0.7 μm (PCL 20%, formic acid, 25 kV, 1.0 mL/h) and 4 μm (PCL 15%, chloroform, 26.5 kV, 0.5 mL/h) were confirmed in FIG. 2B.

As a result, according to FIG. 2B, the structural characteristics of the fibers were visually observed when the nanofiber thickness was 0.7 μm (right column) and 4 μm (left column). Specifically, uniform, bead-free fiber shapes were observed under both nanofiber thickness conditions, suggesting that the electrospinning process of the present invention simultaneously ensures precise control of nanofiber thickness and structural consistency. In particular, the nanofiber sheet with the fiber thickness of 4 μm was found to have high physical strength due to the thick fiber structure thereof, and therefore, the fiber thickness of 4 μm was confirmed as a suitable condition for comparative evaluation of mechanical and tensile properties as a dural substitute.

In addition, the size of pores within the nanofiber sheets having fiber diameters of 0.7 μm and 4 μm and manufactured through the electrospinning process was analyzed in detail. The thickness of the nanofiber, that is, the fiber diameter, is a factor that significantly influences the formation of the porous architecture within the sheet, and the pore size in electrospinning-based nanofiber sheets is determined by the fiber diameter and the inter-fiber spacing.

The experimental results, as shown in FIG. 2B, confirmed the pore size ranged from 0.7 μm to 4 μm upon the application of fiber thicknesses of 0.7 μm and 4 μm. These results suggest that fiber thicknesses of 0.7 μm to 4 μm induce a pore structure that is excellent in terms of cell adhesion and tissue fusion.

As a result, considering the physical properties of the nanofiber assemblies of each sheet illustrated in FIG. 2B, it was confirmed that the dural substitutes, characterized by nanofiber thicknesses of 0.7 μm and 4 μm, can actually exhibit excellent effects in terms of cell adhesion, diffusion, and shape maintenance by featuring the above-described pore sizes. Furthermore, regarding biological functions, it is determined that these dural substitutes, are suitable for evaluating cell adhesion to nanofiber structures, stem cell adhesion rates, and engraftment ability.

In addition, among the dural substitutes, composed of nanofiber assemblies manufactured in Example 1, FIG. 2C shows the results of observing, under magnification, the dural substitutes, having nanofiber thicknesses of 0.7 μm manufactured using each of the polymers PCL and PU.

The results visually confirmed that even under the same electrospinning conditions, the formed nanofiber structures differed depending on the physical properties of the polymers (PCL: semicrystalline, PU: elastic polymer), which may directly affect the biological responsiveness and physical properties of the dural substitute.

Therefore, it was confirmed that by reflecting these inherent polymer properties in the electrospinning design, substitutes centered on cell affinity (PCL-based) or centered on mechanical support (PU-based) can both be manufactured by controlling the polymer properties according to the purpose.

### Example 3. Confirmation of Excellent Adhesion and Engraftment Abilities of Dura-Derived Stem Cells to Dural Substitute

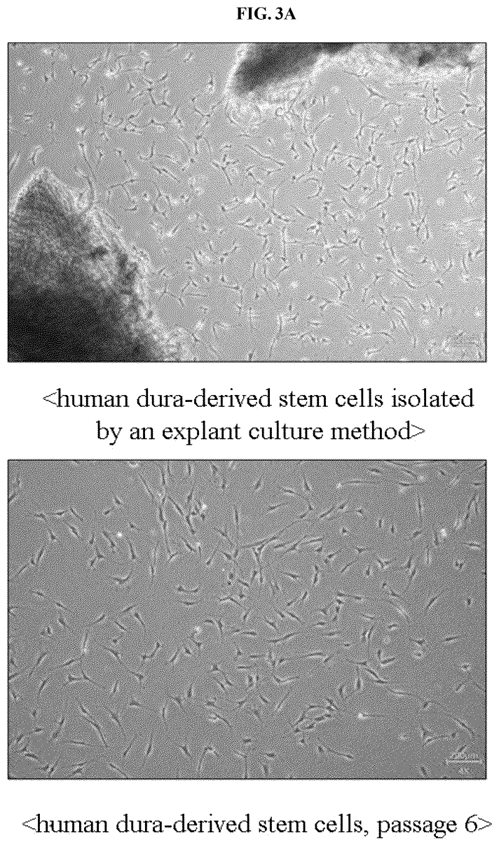

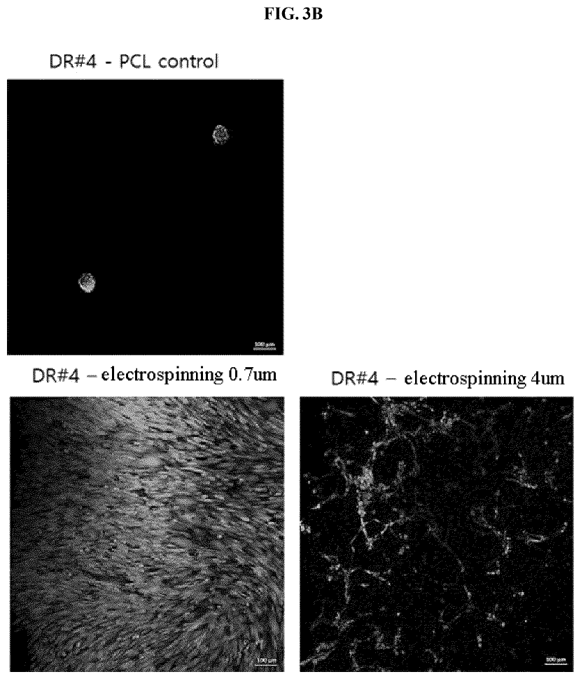

The adhesion of dura-derived stem cells to the electrospun surface of the dural substitute manufactured in Example 1 was analyzed. First, human dura-derived stem cells were prepared for the experiment (FIG. 3A). Specifically, human dura tissue was chopped into the smallest possible size of 1 mm³. Using forceps, the human dura tissue was divided into an amount corresponding to a diameter of approximately 2 mm and placed in a 100 mm cell culture dish, and the tissue was covered with a cover glass. Alpha Minimum Essential Medium Eagle (MEM) complete growth medium (Thermo Fisher, cat. no. 12571-071) containing 10% fetal bovine serum (FBS, GIBCO, cat. no. 16000-044) and 0.1% gentamicin (Sigma, cat. no. G1397) was added, and the cells were cultured in an incubator under 37 °C and 5% CO₂ conditions for up to 14 days. Thereafter, dura mater cells were obtained by filtering the culture medium using a 40 μm strainer (Falcon, cat. no. 352340) and cultured in 100 mm cell culture dishes using Alpha MEM complete growth medium (10% FBS, 0.1% gentamicin). The obtained dura mater cells were cultured again using Alpha MEM complete growth medium containing 10% FBS and 0.1% gentamicin. Subculture was performed using 100 mm cell culture dishes in an incubator under 37 °C and 5% CO₂ conditions. Subculture was initiated when the final cell concentration in each 100 mm cell culture dish was 7×10⁵ cells/mL or higher and the cell confluency was 90% to 95%. After being stocked at P6, the cells were utilized in all experiments.

The electrospun surfaces of the dural substitutes with nanofiber thicknesses of 0.7 μm and 4 μm manufactured in Example 1 were treated with the human dura mater-derived stem cells. At this time, PCL was fabricated into a sheet form by casting rather than electrospinning, and the plate-type printed PCL and the electrospun PCL, both having the outer appearance of a sheet-like shape, were used as controls. As for the stem cell treatment method, the dura mater cells were inoculated onto nanofibers in a 24-well plate at a density of 1×10⁴ cells/well, 1.5 ml/well of alpha MEM complete growth medium (Thermo Fisher, cat. no. 12571-071) containing 10% FBS and 0.1% gentamicin was added. The cells were cultured in an incubator under 37 °C and 5% CO₂ conditions. The culture medium was replaced with a fresh medium every two days, and the cells were cultured for seven days. Human umbilical vein endothelial cells (HUVECs, Lonza, cat.no. C2517A) were inoculated at a density of 1×10⁴ cells/well into a 24-well plate containing the dura mater cells already cultured on nanofibers, and co-cultured with 1.5 ml/well of Endothelial Cell Growth Medium 2 (PrompCell, cat.no. C-22011) supplemented with premixed supplements. The culture medium was then replaced with a fresh medium every two days, and the cells were cultured for three weeks in an incubator under 37 °C and 5% CO₂ conditions.

As a result, when the human dura-derived stem cells were attached to electrospun surfaces with nanofiber thicknesses of 0.7 μm and 4 μm, excellent adhesion was observed at both thicknesses, and the engraftment ability of the attached stem cells was maintained at an excellent level. These results confirmed that a nanofiber sheet having a thickness in a range including the two above-mentioned thicknesses can be effectively utilized as a dural substitute for cell transplantation and tissue regeneration (FIG. 3B). In particular, it was confirmed that when the dura-derived stem cells and human umbilical vein endothelial cells were co-cultured on the dural substitute with a nanofiber thickness of 0.7 μm, both cells exhibited excellent adhesion and proliferation abilities with high overall cell viability.

### Example 4. Confirmation of Excellent Angiogenesis Ability in Dura-Derived Stem Cells Attached to Dural Substitute

Example 3 confirmed that when the dural substitute was treated with the dura-derived stem cells, excellent adhesion and engraftment abilities were exhibited.

In this example, to confirm whether angiogenesis occurred in the dura-derived stem cells on the electrospun surface of the dural substitute manufactured in Example 1, the expression of vascular-related markers was analyzed after co-culture with HUVECs. Specifically, after co-culture of the dura-derived stem cells and HUVECs in a 24-well plate, the attached nanofibers were separated, transferred to a new 24-well plate, washed twice with 1 ml/well of Dulbecco's phosphate-buffered saline (DPBS, Thermo Fisher, cat. no. 14190144), and then fixed with 2% paraformaldehyde (PFA) for 20 minutes at room temperature. Next, the samples were washed three times with 1.5 ml/well of DPBS, and non-specific binding was blocked for one hour with normal goat serum blocking solution (NGS, Vector Laboratories, cat.no. S-1000). Thereafter, primary antibodies against the vascular endothelial cell marker CD31 (rabbit, 1:200, Invitrogen) and the angiogenic factor vascular endothelial growth factor (VEGF, mouse, 1:100, Santa Cruz) were allowed to react on the nanofibers at 4 °C for 24 hours under light-blocked conditions. After the primary antibody reaction, the cells were washed three times with a 0.1 M phosphate buffer (PB, DYNEBIO, cat.no. CBP3373) for five minutes at room temperature, and then allowed to react with Alexa-conjugated anti-rabbit (Alexa Fluor 488-rabbit, A11008, Invitrogen, USA) and anti-mouse (Alexa Fluor 546-mouse, A11003, Invitrogen, USA) at room temperature for two hours under light-blocked conditions. Finally, the cell nuclei were stained with 4′,6-diamidino-2-phenylindole (DAPI) at room temperature and fixed by placing a cover slide using 200 μm of mounting medium (Invitrogen, Fluoromount-G™ Mounting Medium cat.no. 15586276), and the expression of angiogenesis-related markers was analyzed using a confocal microscope (LSM 900, Carl Zeiss, USA).

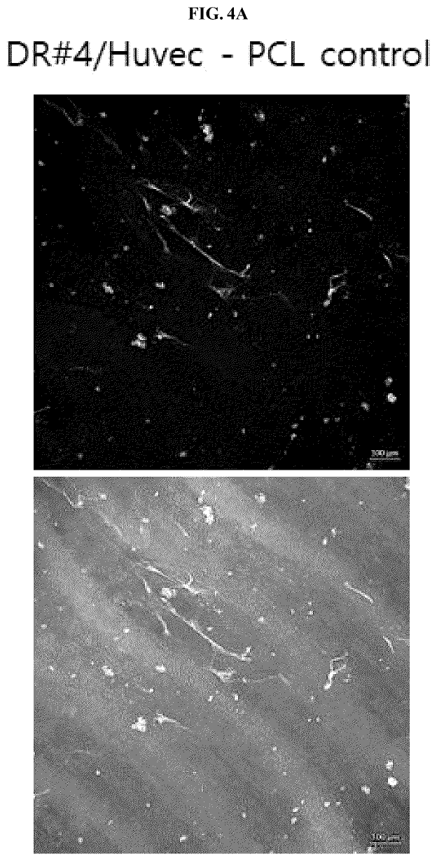

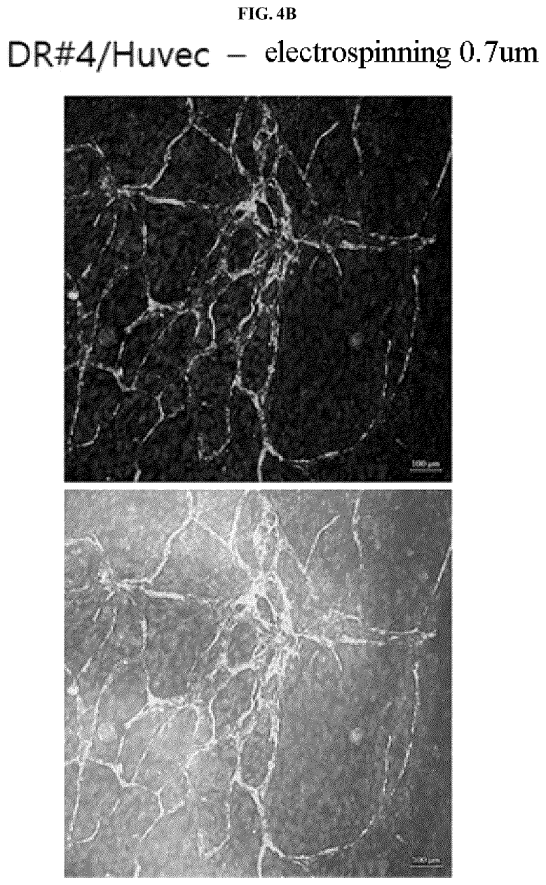

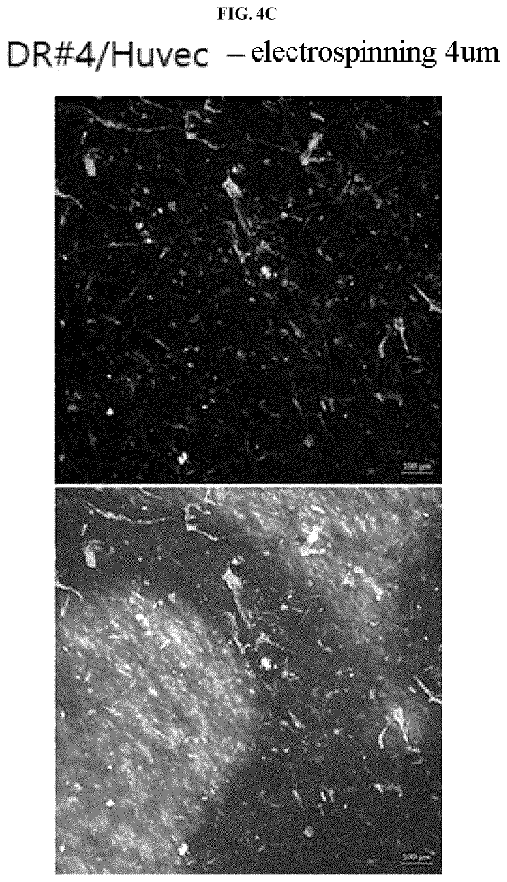

As a result, when the human dura-derived stem cells attached to the electrospun surfaces of the dural substitutes with various nanofiber sizes were co-cultured with the HUVECs, blood vessel formation was observed within two weeks in the dural substitute having a fiber thickness of 0.7 μm and also in the dural substitute having a fiber thickness of 4 μm (FIGS. 4A to 4C).

In addition, the observation of the expression levels of CD31, a vascular endothelial cell-specific marker, showed that when the nanofiber thickness was 0.7 μm, effective interactions between the two types of cells were induced, forming a dense vascular network, and these results demonstrated superior potential for tissue engineering-based vascular regeneration. Furthermore, experiments confirmed that vascular structures were effectively formed from the attached stem cells even when the nanofiber thickness was 4 μm. The formed vascular structures were found to maintain a stable form and provide an environment necessary for cell survival and tissue regeneration.

In conclusion, when the thickness of the nanofiber was 0.7 μm to 4 μm, the pore size of the nanofiber assembly was also 0.7 μm to 4 μm, which could induce excellent adhesion of the dura mater-derived stem cells. As a result, it was found that not only is the engraftment ability of the attached stem cells significantly superior, but the formation of vascular structures within the cells also contributes to cell survival and nutrient supply within the tissue. Therefore, it was confirmed that the dural substitute of the present invention can be effectively applied in the fields of tissue engineering and regenerative medicine.

The foregoing description of the present invention is intended for illustrative purposes, and it will be understood by those skilled in the art that various modifications can be made thereto in other specific forms without departing from the technical spirit or essential features of the present invention. Therefore, the embodiments described above should be understood as illustrative in all respects and not restrictive.

1. A method of manufacturing a dura replacement, comprising:
S1) a step of preparing a composition for manufacturing a dura replacement by dissolving a polymer in a solvent and manufacturing a dura replacement by electrospinning the composition; and
S2) a step of treating the electrospun surface of the dura replacement with dura-derived stem cells,
wherein the dura replacement is a nanofiber assembly.

2. The method of claim 1, wherein the thickness of the nanofiber and the pore size within the nanofiber are 0.1 μm to 10 μm.

3. The method of claim 1, wherein the nanofiber assembly is any one selected from the group consisting of a nanofiber sheet, a nanofiber mat, a nanofiber film, a nanofiber membrane, a nanofiber web, a nanofiber textile, and a nanofiber fabric.

4. The method of claim 1, wherein the polymer is any one selected from the group consisting of polycaprolactone (PCL), polyurethane (PU), a poly(lactic-co-glycolic acid) (PLGA) copolymer, polylactic acid (PLA), poly(3-hydroxybutyrate-co-3-hydroxyvalerate (PHBV), polyvinyl alcohol (PVA), collagen, gelatin, elastin, chitosan, silk fibroin, alginate, poly(glycolic acid) (PGA), poly(L-lactic acid) (PLLA), poly(L-lactic acid)/collagen (PLLA/CL), and polyethylene oxide (PEO).

5. The method of claim 1, wherein the solvent is any one selected from the group consisting of formic acid, chloroform acid, acetic acid, acetone, dichloromethane, 1,2-dichloroethane, dimethylformamide, ethanol, isopropyl alcohol, water, and tetrahydrofuran.

6. The method of claim 1, wherein the electrospinning is characterized by the following conditions:
a voltage of 1 to 30 kV;
an output rate of 0.1 to 5 mL/h; and
a distance of 1 to 20 cm between a nozzle and a collector.

7. The method of claim 1, further comprising S3) a step of treating the electrospun surface of the dura replacement with an angiogenic cell.

8. The method of claim 7, wherein the angiogenic cell is any one selected from the group consisting of a stem cell, an induced pluripotent stem cell, a vascular endothelial cell, and an umbilical cord vein endothelial cell.

9. A kit for manufacturing a dura replacement, comprising a composition for manufacturing a dura replacement and instructions,
wherein the composition comprises a polymer and a solvent as active ingredients, wherein the dura replacement is a nanofiber assembly, and
wherein the instructions teach the method of claim 1.
